(19) United States
(12) Patent Application Publication
(10) Pub. No.: US 2021/0246365 A1
(43) Pub. Date: Aug. 12, 2021

(54) METHOD OF MONITORING A FLUID, USE OF A TRACER, AND TRACER COMPOSITION

(71) Applicant: Johnson Matthey Public Limited Company, London (GB)

(72) Inventors: Neil BORRELL, Billingham (GB); Aidan BRIERLEY, Billingham (GB); Thomas William BURNS, Billingham (GB); Kirk BUTLER, Billingham (GB); Hannah PALMER, Billingham (GB); Carl Robert TIPTON, Billingham (GB)

(21) Appl. No.: 17/054,722

(22) PCT Filed: Jun. 27, 2019
(86) PCT No.: PCT/GB2019/051810

§ 371 Date: Nov. 11, 2020

(30) Foreign Application Priority Data
Jul. 4, 2018 (GB) 1810936.3

(51) Int. Cl. C09K 8/84; C09K 11/65; E21B 49/08; G01N 21/64; G01N 33/28

(52) U.S. Cl. CPC C09K 8/845; C09K 11/65; B82Y 20/00; G01N 21/64; G01N 33/2882; E21B 49/087

(57) ABSTRACT

A method of monitoring a fluid is described comprising: introducing a tracer into the fluid and analysing the fluid to determine if the tracer is present in the fluid; characterised in that the tracer comprises luminescent carbon-based nanoparticles exhibiting a peak luminescence intensity at an emission wavelength of at least 500 nm. The method is in particular a method of monitoring a parameter of a hydrocarbon well, pipeline or formation. A use of a tracer and a tracer composition comprising carbon-based nanoparticles exhibiting a peak luminescence intensity at an emission wavelength of at least 500 nm are also described.

### FIELD OF THE INVENTION

The present invention relates to the use of nanoparticle tracers in monitoring of fluids. The invention in particular applies to the use of nanoparticle tracers in monitoring of hydrocarbon wells, pipelines or formations, and to methods of monitoring hydrocarbon wells, pipelines or formations, but may also find application in process diagnostics and other areas where the use of a tracer or taggant composition may be applicable. The invention additionally applies to tracer compositions for such use in such methods. More specifically, but not exclusively, the invention relates to the use of nanoparticle tracers for monitoring produced and injected water from different zones of hydrocarbon wells and to methods of monitoring the same.

### BACKGROUND

The use of tracers to monitor aspects of the performance of hydrocarbon wells is an established technique. The tracers may be water tracers, in that they are predominantly soluble or dispersible in water, oil tracers, in that they are soluble or dispersible in the hydrocarbons in the formation, or partitioning tracers, in that they are soluble or dispersible between both the water and hydrocarbon phases. Some tracing methods will employ more than one type of tracer and use the difference in behaviour to deduce properties of the hydrocarbon formation. For example, partitioning and water tracers may be injected into a production well along with injected water and then monitored as they are subsequently produced from the well. The time difference between the production of the water tracers, which are produced with the returning injected water, and the partitioning tracers, whose production is delayed by their interaction with the hydrocarbons in the formation, can be used to deduce parameters relating to the local remaining hydrocarbon content of the formation. Alternatively, applications may use only water tracers. For example, water tracers may be introduced in an injection well and their presence monitored at adjacent production wells in order to obtain information about the flux of water from the injection well to the production well. In addition to injected techniques, it is also known to introduce tracers into a well by including them in articles placed into the well. By detecting the rate of tracer production over time, information can be deduced about performance of the hydrocarbon well.

In order to be useful as a tracer, a compound should be thermally stable in that it should be stable at the temperatures typically encountered in hydrocarbon wells, which may be 60 to 90° C. Desirably, a tracer is stable in temperatures up to maybe 160 or 180° C. so as to permit use in high temperature hydrocarbon wells. For a water tracer, the compound should be highly selective toward water over oil and will preferentially disperse in water over oil. The compound should also be detectable in small to very small quantities, for example at levels below 100 ppb, preferably at levels of 50 ppb or lower, more preferably at levels of 10 ppb or lower, and most preferably in the parts per trillion (ppt) range (that is, at levels less than 1 ppb). The levels are determined on a mass/mass basis. The compound should also be environmentally acceptable with low toxicity, for inserting into the ground, but also not a compound that is naturally present in the ground in such quantities as to contaminate the results of the tracer study.

Typical detection methods include gas chromatography—mass spectrometry (GC-MS), gas chromatography—mass spectrometry—mass spectrometry (GC-MS-MS), liquid chromatography—mass spectrometry (LC-MS), liquid chromatography—mass spectrometry—mass spectrometry (LC-MS-MS) and high-pressure liquid chromatography (HPLC), which can typically detect very low concentrations of the tracers in the produced fluids. It is desirable that tracers should be detectable in low quantities and also that they can be reliably distinguished from other tracers.

Tracers may comprise or include a luminophore (that is, a material that can emit energy upon excitation with energy) and a presence of the tracer may be determined by optical spectroscopy of that emission. Known luminophores include fluorophores, that is, materials that exhibit fluorescence. However, known fluorophores may have undesirable properties relating to their performance as tracers or to their environmental impact. For example, metallic quantum dots are frequently toxic and fluorescent dyes tend to be unstable in hydrocarbon well conditions.

Luminescent and for example fluorescent nanoparticles have attracted recent attention for such application. Examples include semiconductor quantum dots, metallic quantum dots, carbon dots, and other carbonaceous nanomaterials.

Carbon dots are small carbon-based nanoparticles (for example less than 100 nm in size, and generally less than 10 nm in size) which have been found to exhibit useful luminescent properties and in particular fluorescent properties together with low toxicity and high chemical stability that make them potentially attractive for tracer applications. Carbon dots are also known in the literature as carbon quantum dots, C-dots, carbon nanoparticles, amorphous carbon dots, graphitic carbon dots, graphene quantum dots or graphene dots. Novel carbon quantum dot (CQD-) based fluorescent tracers have been proposed for production and well monitoring. They may be structured or have surface modifications to exhibit high dispersibility in water. Their use as aqueous phase tracers has been discussed for example in U.S. Pat. No. 9,891,170.

Produced fluid from hydrocarbon wells generally contains organic species which are naturally fluorescent. This fluorescence tends to be exhibited at the blue end of the visible spectrum, overlapping with that of carbon-based nanoparticles dots such as are described in U.S. Pat. No. 9,891,170, which exhibit a peak fluorescence intensity occurring at an emission wavelength of for example 440 to 475 nm. This may limit the effective use of such carbon dots without significant fluid preparation, separation and cleaning, such as is described in U.S. Pat. No. 9,891,170.

These organic species which are naturally fluorescent in the relevant wavelength range are not limited to the oil phase. Some of the organic species exhibit appreciable water solubility and may be present in produced water. As a consequence, even complete separation of oil and water phases in the produced fluid will not prevent this effect.

Metallic quantum dots are known that fluoresce at longer wavelengths less likely to overlap with the fluorescence wavelengths of residual organics but these are typically based on heavy metals such as lead and cadmium and their use as tracers would raise environmental issues.

The present invention seeks to overcome one or more of the above disadvantages of the prior art. In particular, preferred embodiments of the present invention seek to provide improved carbon-based nanoparticle tracers for use in hydrocarbon well monitoring and in particular for use in monitoring produced water from hydrocarbon wells.

### SUMMARY OF INVENTION

According to an aspect of the invention, there is provided use of a tracer in monitoring a fluid wherein the tracer comprises a plurality of luminescent carbon-based nanoparticles exhibiting a peak luminescence intensity at an emission wavelength of at least 500 nm, more preferably at least 600 nm, preferably no more than 1500 nm, and for example in the range 500 or 600 to 1500 nm.

A particular preferred use is use in monitoring a parameter of a hydrocarbon well, pipeline or formation, and discussion herein considers such use by way of example, but other uses for example in process diagnostics and other areas where the use of a tracer or taggant composition may be encompassed within the scope of the invention.

A method of monitoring a fluid is also provided, the method comprising:
introducing a tracer into the fluid; and
analysing the fluid to determine an amount of tracer present in the fluid;
   characterised in that the tracer comprises carbon-based nanoparticles exhibiting a peak fluorescence intensity at an emission wavelength of at least 500 nm, more preferably at least 600 nm and for example in the range 600 to 1500 nm and more preferably in the range 600 to 900 nm.

According to one particularly preferred method there is provided a method of monitoring a parameter of a hydrocarbon well, pipeline or formation, the method comprising:
introducing a tracer into the hydrocarbon well, pipeline or formation;
producing a fluid from the hydrocarbon well, pipeline or formation; and
analysing the fluid to determine an amount of tracer present in the fluid and determine a parameter of the hydrocarbon well, pipeline or formation based on the amount of tracer present in the fluid;
wherein the fluid comprises produced water from which an oil phase has been largely removed but which fluid still comprises organic species which are naturally fluorescent at a wavelength of below 500 nm, and the tracer comprises a plurality of luminescent carbon-based nanoparticles exhibiting a peak luminescence intensity at an emission wavelength of at least 500 nm, preferably in a range 500 nm to 1500 nm.

The aforementioned method is applicable for applications in which there are organic species which are naturally fluorescent at wavelengths below 500 nm and which organic species exhibit appreciable water solubility. The method allows the use of luminescent carbon-based nanoparticles as tracers without requiring significant fluid preparation, separation and cleaning to remove the organic species which are naturally fluorescent at wavelengths below 500 nm.

A tracer composition is also provided for use in such methods, the tracer composition including a tracer comprising carbon-based nanoparticles exhibiting a peak fluorescence intensity at an emission wavelength of at least 500 nm, more preferably at least 600 nm and for example in the range 600 to 1500 nm and more preferably in the range 600 to 900 nm.

### DESCRIPTION OF THE DRAWINGS

Embodiments of the present invention will now be described, by way of example, and not in any limitative sense, with reference to the accompanying drawings, of which:

FIGS. 1 to 3 are a series of comparative spectra comparing the fluorescence intensity response of the fluorescent organic species found in produced water with that of a prior art carbon-based nanoparticle tracer and that of carbon-based nanoparticle tracer for use in an embodiment of the invention; and

FIG. 4 shows the three spectra of FIGS. 1 to 3 superimposed.

### DETAILED DESCRIPTION

Embodiments of the invention as described herein are directed to a tracer based on the use of luminophores, wherein the luminophores comprise carbon-based nanoparticles. In the context of this invention a luminophore is a material that emits light by luminescence (that is, a material that can emit light upon light excitation) by a mechanism that may include without limitation fluorescence and phosphorescence.

Carbon-based nanoparticles are considered to emit light by fluorescence, and references to emission of light or luminescence may accordingly in the preferred case be construed to be references to fluorescence, references to a peak emission intensity likewise being in the preferred case a peak fluorescence intensity. However, the invention is not limited to a particular mechanism but encompasses any emission by carbon-based nanoparticles exhibiting a peak intensity at an emission wavelength in the indicated ranges. In particular, the invention may additionally encompass carbon-based nanoparticles which exhibit phosphorescence, for example by compositional modification or surface modification, in addition to or as an alternative to fluorescence.

Advantageously, the use of carbon-based nanoparticles with a peak luminescence intensity and in the preferred case a peak fluorescence intensity that occurs at an emission wavelength of at least 500 nm means that the tracer luminesces in a different part of the spectrum to many if not most of the organic species which may be present. It may be less necessary to prepare the produced fluid to remove such species before looking for the presence of the tracer.

In many applications, it may be advantageous for the carbon-based nanoparticles to exhibit a narrow emission bandwidth within the indicated preferred range of wavelength of peak luminescence intensity. For example, in preferred case the carbon-based nanoparticles exhibit an emission bandwidth with a full width half maximum of no more than 200 nm and more preferably of less than 50 nm.

Optionally, a tracer in accordance with the invention may comprise carbon-based nanoparticles exhibiting more than one wavelength of peak luminescence intensity, so long as the tracer comprises nanoparticles exhibiting at least one wavelength of peak luminescence intensity in the desired range.

Tracers of the invention can be used in conjunction with at least one other tracer or class of tracer. Such other tracer may for example be a water dispersible or oil dispersible tracer. Such other tracer may be a nanoparticle tracer or may be a tracer that is not a nanoparticle tracer.

For example, the other tracer can be one used for enhanced oil recovery or to monitor hydrocarbon wells, pipelines, formations.

In another example, the other tracer can be one used for any other application in process diagnostics and other areas where the use of a tracer or taggant composition may be applicable.

The other tracer may be one used to track the movement of a well treatment agent. An example well treatment agent can be a corrosion inhibitor.

The wavelength of peak luminescence intensity may be modified by doping. The carbon-based nanoparticles may be doped. A large range of potential dopants is available. The carbon-based nanoparticles may be doped by addition of one or more metal species. The carbon-based nanoparticles may be doped by addition of one or more non-metallic species. For example, the carbon-based nanoparticles may be doped by addition of one or more of nitrogen, sulfur, boron, silicon, fluorine, selenium, titanium, magnesium, bismuth and phosphorus to form nitrogen-doped, sulfur-doped, boron-doped, silicon-doped, fluorine-doped, selenium-doped, titanium-doped, magnesium-doped, bismuth-doped and phosphorus-doped carbon-based nanoparticles, respectively. Techniques for preparing carbon-based nanoparticles with such dopants are known.

Any suitable fabrication technique may be used. The fabrication of the carbon-based nanoparticles is generally either by the breaking down of larger carbonaceous structures such as nanodiamonds, graphite, carbon nanotubes, graphene sheets, carbon soot and the like by methods including arc discharge, laser ablation, sonication, chemical ablation, electrochemical carbonization and microwave irradiation; or by synthesis from molecular precursors by methods including combustion/thermal treatments, supported synthetic, sonication and microwave synthetic routes etc.

A known method of forming carbon-based nanoparticles suitable for use in accordance with the invention is to provide an electrochemical cell including at least one graphite electrode and an electrolyte which may comprise another unique carbon source. A current is applied across electrodes of the electrochemical cell to form carbon-based nanoparticles comprising carbon from the carbon source.

Another known method of forming carbon-based nanoparticles suitable for use in accordance with the invention is to make use of microwave irradiation to thermally heat a solution of molecular precursors.

Another known method of forming carbon-based nanoparticles suitable for use in accordance with the invention is to make use of a hydrothermal or solvothermal technique to heat a solution of molecular precursors.

Preferably the tracer is a water tracer. Preferably therefore, the carbon-based nanoparticles comprising the tracer are water soluble or water dispersible. In a possible embodiment, at least a part of the surface of the carbon-based nanoparticles is hydrophilic and/or oleophobic. For example, at least a part of the surface of the carbon-based nanoparticles comprises hydrophilic groups, for example selected from one or more of: amine groups, hydroxyl groups, carbonyl groups. Additionally, or alternatively the outer surface may be otherwise functionalized to improve stability and/or the luminescent properties. Techniques for modifying the surface of carbon-based nanoparticles to give such functionality are known.

Thus, the use may involve monitoring the flow and/or movement of water through or from a well or formation. For example, the use may determine the source of produced water by introducing the tracer into a defined part of the well or formation and monitoring for the presence of the tracer in produced water. As another example, the use may involve a partitioning study to determine residual oil saturation where the tracer is used as the conservative, water soluble tracer. As another example, the use may involve determining the presence or absence of a well treatment agent which had previously been tagged.

In a possible alternative application however, the tracer is an oil tracer. In such a case, the carbon-based nanoparticles comprising the tracer are soluble or dispersible in the oil phase. In a possible embodiment, at least a part of the surface of the carbon-based nanoparticles is hydrophobic and/or oleophilic. For example, at least a part of the surface of the carbon-based nanoparticles comprises hydrophobic groups and/or the surface is otherwise functionalized to improve stability and/or the luminescent properties.

Tracers of the invention may have sufficient thermal stability to survive the conditions in a hydrocarbon well. Such tracers may also be detectable, for example using GC-MS, in very low concentrations, for example concentrations of 10 ppb or less, preferably concentrations of 1 ppb or less, more preferably concentrations of 100 ppt or less, yet more preferably concentrations of 10 ppt or less and still more preferably concentrations of 1 ppt or less. The tracers may be fabricated to show a high selectivity towards water instead of oil. Thus, the tracer may be a water tracer. The tracer may have a log P value of less than −1. The log P value is a well-known value for characterising the partitioning preference of a compound for water or oil. The value is the log of the ratio of the equilibrium concentration of a species in oil (octanol) to the equilibrium concentration of the species in water. Thus, the concentration of the tracer in water is preferably at least 10 times, and more preferably at least 100 times, that of the tracer in oil.

The parameter monitored by use of the tracer may be a parameter related to a property, such as flow or composition, of the well, pipeline or formation and may be an absolute parameter or a relative parameter. A relative parameter may describe a property of one part of the well, pipeline or formation relative to another part. Examples of parameters that may be monitored include a relative distribution of water production along a lateral or between laterals in multiple interconnected well systems, a formation fluid composition, or a measure of rock heterogeneity. Preferably, the parameter relates to a well or formation. It will be appreciated that when a parameter is said to relate to a well or formation, that well refers to the constructed apparatus for extracting the hydrocarbon, while formation refers to the natural structure in which the hydrocarbon is located and from which it is extracted via the well.

There is provided a method of monitoring a fluid, the method comprising:
introducing a tracer into the fluid; and
analysing the fluid to determine an amount of tracer present in the fluid;
   characterised in that the tracer comprises carbon-based nanoparticles exhibiting a peak fluorescence intensity at an emission wavelength of at least 500 nm, more preferably at least 600 nm and for example in the range 600 to 1500 nm and more preferably in the range 600 to 900 nm.

A particular preferred application of the method is in monitoring a parameter of a hydrocarbon well, pipeline or formation.

In such a case the invention provides a method of monitoring a parameter of a hydrocarbon well, pipeline or formation, the method comprising:
introducing a tracer into the hydrocarbon well, pipeline or formation;
producing a fluid from the hydrocarbon well, pipeline or formation; and
analysing the fluid to determine an amount of tracer present in the fluid;
   characterised in that the tracer comprises carbon-based nanoparticles exhibiting a peak fluorescence intensity at an emission wavelength of at least 500 nm, more preferably at least 600 nm and for example in the range 600 to 1500 nm and more preferably in the range 600 to 900 nm.

However, the method may find other application for example in process diagnostics and other areas where the use of a tracer or taggant composition may be useful.

The step of determining the amount of tracer present in the fluid encompasses either determining whether a tracer is present or determining a quantity of the tracer present or both.

Preferably the tracer is a water tracer. The fluid produced may therefore comprise water. Produced fluids from a hydrocarbon well, pipeline or formation may comprise a mixture of hydrocarbon and water. Thus, the method may involve producing a fluid comprising water and for example a mixture of hydrocarbon and water from the hydrocarbon well, pipeline or formation; and analysing the produced fluid to determine an amount of the tracer present in the fluid.

Produced fluids from a hydrocarbon well, pipeline or formation may comprise a mixture of an oil phase and a water phase. Typically, these phases may be separated before tracer analysis is performed. Preferably the fluid comprises a produced water phase from which the oil phase has been largely removed. Thus, the method may involve producing a fluid comprising produced water from which the oil phase has been largely removed, for example being a fluid in which the oil phase comprises no more than 10% by volume, more preferably no more than 1% by volume, and for example consisting essentially of produced water from which the oil phase has been substantially entirely removed; and analysing the produced fluid to determine an amount of the tracer is present in the fluid.

Even in a produced water phase from which the oil phase has been largely removed, water soluble organic species which are naturally fluorescent in the relevant wavelength range may be present in quantity in the produced water. The use of tracers in accordance with the invention as water tracers in such a case accordingly confers the aforementioned advantages.

According to one particularly preferred method there is provided a method of monitoring a parameter of a hydrocarbon well, pipeline or formation, the method comprising:
introducing a tracer into the hydrocarbon well, pipeline or formation;
producing a fluid from the hydrocarbon well, pipeline or formation; and
analysing the fluid to determine an amount of tracer present in the fluid and determine a parameter of the hydrocarbon well, pipeline or formation based on the amount of tracer present in the fluid;
wherein the fluid comprises produced water from which an oil phase has been largely removed but which fluid still comprises organic species which are naturally fluorescent at a wavelength of below 500 nm, and the tracer comprises a plurality of luminescent carbon-based nanoparticles exhibiting a peak luminescence intensity at an emission wavelength of at least 500 nm, preferably in a range 500 nm to 1500 nm.

The aforementioned method is applicable for applications in which there are organic species which are naturally fluorescent at wavelengths below 500 nm and which organic species exhibit appreciable water solubility. The method allows the use of luminescent carbon-based nanoparticles as tracers without requiring significant fluid preparation, separation and cleaning to remove the organic species which are naturally fluorescent at wavelengths below 500 nm.

The method may monitor a parameter of a hydrocarbon well or formation. The tracer may be introduced into the well by any method. For example, the introducing may comprise injecting the tracer into the well or formation. For example, the tracer may be injected into the well or formation of which the parameter is being monitored. The tracer may be injected into an adjacent well or formation and thus be introduced into the formation via the adjacent well or formation. The tracer may be introduced into the well or formation during construction of the well. For example, the tracer may be provided comprised in a solid article incorporated into or attached to a component part of the well, such as a filter, mesh, sand screen, in-flow control device or valve. The tracer may be introduced into the well or formation as a liquid, for example in solution or as an emulsion with injection fluid, such as drilling fluids, hydraulic fracturing fluids or injection water. The tracer may be introduced into the well as a solid, for example as slurry with drilling fluids, hydraulic fracturing fluids or injection water, or as a solid or liquid encapsulated in another solid. The tracer may be introduced into the well or formation by introducing a proppant which comprises the tracer.

The analysing may be performed on-line, at-line or off-line. In the latter cases, samples of the fluid may be taken and transferred to a laboratory, either at the drilling location (at-line) or at a remote location (off-line) for analysis. Preferably the analysis is carried out using spectroscopy. An advantage of the method of the invention may be that the tracer may be readily distinguishable from prior art tracers, many of which now already contaminate a large number of hydrocarbon wells, using GC-MS.

The analysis may be qualitative, in that it determines whether the tracer is present or not; or it may be quantitative in that it determines if the tracer is present by determining the level, for example the concentration, of the tracer in the fluid; or it may be semi-quantitative in that by using the production rates it determines the relative flow from different regions of the hydrocarbon well. Preferably the analysis determines the level at which the tracer is present in the fluid. The level may be determined as a ratio of parts of tracer per part of fluid for example. Thus, the method may comprise determining the concentration of the tracer in the fluid.

According to another aspect of the invention there is provided a tracer composition including a tracer comprising carbon-based nanoparticles exhibiting a peak fluorescence intensity at an emission wavelength of at least 500 nm, more preferably at least 600 nm and for example in the range 600 to 1500 nm and more preferably in the range 600 to 900 nm.

Preferably the tracer composition is a water tracer.

It will be appreciated that features described in relation to one aspect of the invention may be equally applicable in another aspect of the invention. For example, features described in relation to the use of the tracer of the invention may be equally applicable to the method of the invention, and vice versa. Features described in relation to the tracer composition may be equally applicable to the method or the use, and vice versa. The skilled person will realise where some features may not be applicable to, and may be excluded from, particular aspects of the invention.

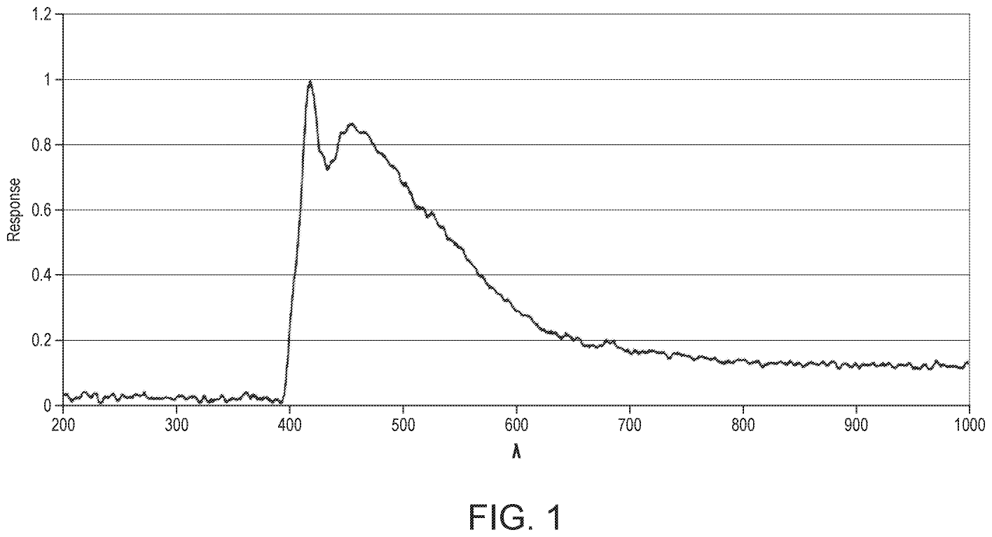

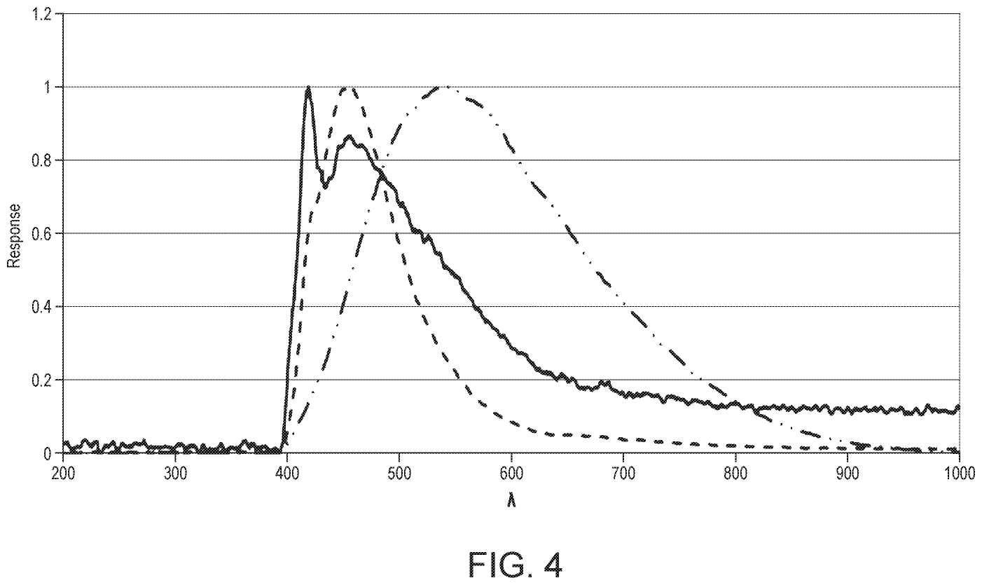

FIGS. 1 to 3 are a series of comparative spectra comparing the fluorescence intensity response of the fluorescent organic species found in produced water with that of a prior art carbon-based nanoparticle tracer and that of carbon-based nanoparticle tracer for use in an embodiment of the invention. FIG. 4 shows the three spectra of FIGS. 1 to 3 superimposed.

For each of FIGS. 1 to 4, emission wavelength in nm on the x-axis is plotted against normalised response intensity. FIGS. 1 to 3 show respective fluorescence response intensity spectra for: produced water, a prior art carbon-based nanoparticle tracer, and a carbon-based nanoparticle tracer illustrative of an embodiment of the invention.

FIG. 1 shows an intensity spectrum for a sample of produced water. The sample is one from which at least 99% of the oil phase has been removed. Even so, there is strong fluorescence from organics which have distributed into and for example dissolved in the produced water phase. The peak region of fluorescence is in particular found to occur at shorter wavelengths in the visible spectrum. Only limited fluorescence is exhibited above 500 nm, even less above 550 nm, and almost none beyond 600 nm.

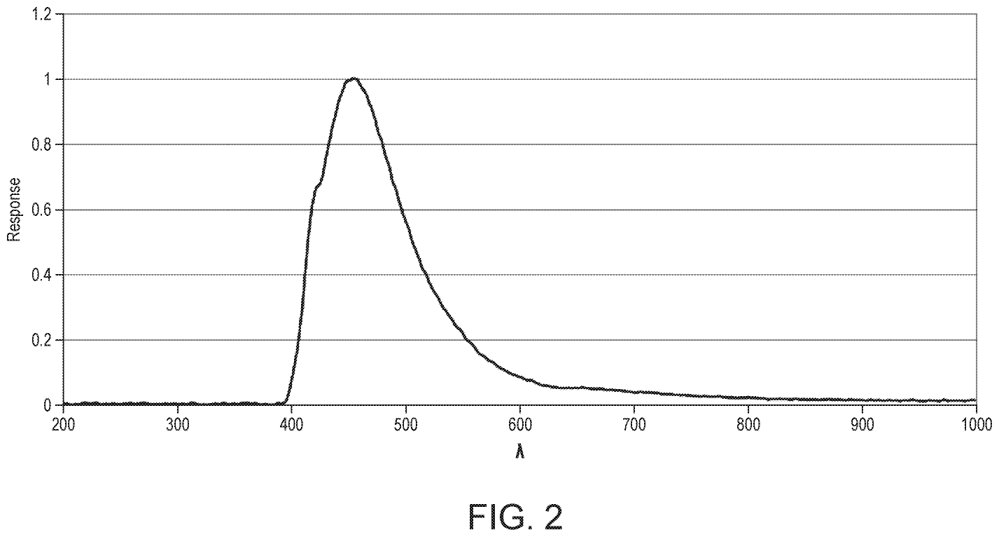

In FIG. 2 a comparable spectrum is shown for a prior art carbon-based nanoparticle tracer having a peak fluorescence intensity at the blue end of the visible spectrum. As can be seen, this exhibits strong fluorescence in the blue/cyan end of the spectrum, with most fluorescence occurring in the range 450-520 nm.

It can be seen that a major part of the intensity of the background fluorescence overlaps with the peak fluorescence intensity of the tracer of FIG. 2. This is shown particularly by the superimposed representations in FIG. 4. In practical use therefore, it may become hard to distinguish the fluorescence of the tracers and the fluorescence of the residual organics.

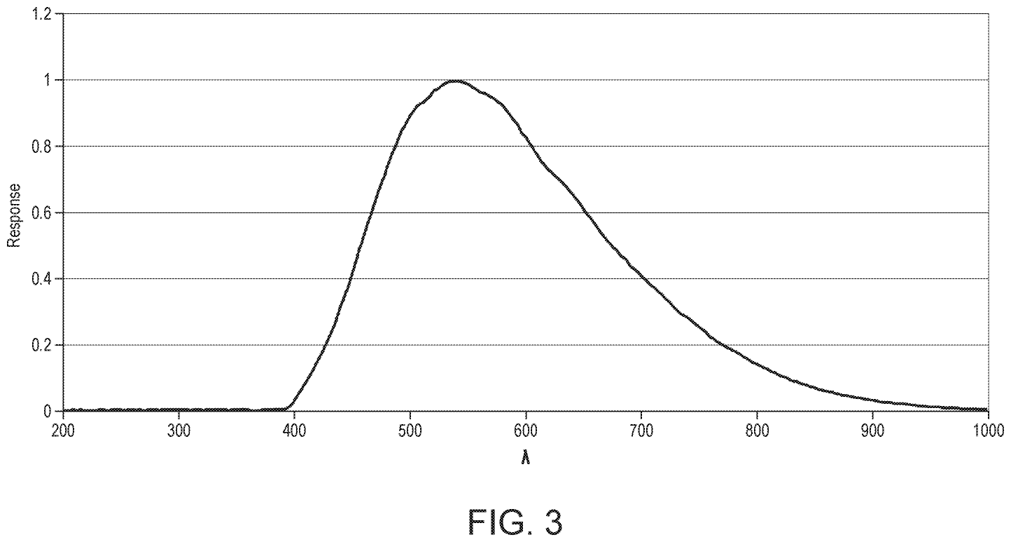

In FIG. 3 a comparable spectrum is shown for a carbon-based nanoparticle tracer illustrative of an embodiment of the invention which has been produced by microwave synthesis. This produces a peak emission intensity at around 550 nm, and still exhibits significant fluorescence in the 600-1500 nm range.

FIG. 4 provides a comparison of these spectra with them superimposed on the same axes, the FIG. 1 spectrum shown by the solid line, the FIG. 2 spectrum by the dashed line, and the FIG. 3 spectrum by the dot-dashed line.

A comparison of the FIG. 2 tracer spectrum with the FIG. 1 background shows a significant degree of overlap. A comparison of the FIG. 3 tracer spectrum with the FIG. 1 background shows much less overlap. Thus, fluorescence attributable to the tracer of FIG. 3 is much more effectively distinguished from that attributable to the residual organics shown in FIG. 1 than is the fluorescence attributable to the tracer of FIG. 2.

The material of FIG. 3 offers the potential for improved carbon-based nanoparticle tracers for use in hydrocarbon well monitoring and in particular for use in monitoring produced water from hydrocarbon wells as it exhibits a fluorescence that can be more readily identified even in the presence of the fluorescence attributable to other materials such as residual organics present in the produced water.

1-29. (canceled)

30. A method of monitoring a parameter of a hydrocarbon well, pipeline or formation, the method comprising:
introducing a tracer into the hydrocarbon well, pipeline or formation;
producing a fluid from the hydrocarbon well, pipeline or formation; and
analyzing the fluid to determine an amount of tracer present in the fluid and determine a parameter of the hydrocarbon well, pipeline or formation based on the amount of tracer present in the fluid;
wherein the fluid comprises produced water from which an oil phase has been largely removed but which fluid still comprises organic species which are naturally fluorescent at a wavelength of below 500 nm, and the tracer comprises a plurality of luminescent carbon-based nanoparticles exhibiting a peak luminescence intensity at an emission wavelength of at least 500 nm.

31. A method according to claim 30, wherein the tracer comprises a plurality of carbon-based nanoparticles exhibiting a peak luminescence intensity at an emission wavelength of at least 600 nm.

32. A method according to claim 31, wherein the tracer comprises a plurality of carbon-based nanoparticles exhibiting a peak luminescence intensity at an emission wavelength in the range 600 nm to 1500 nm.

33. A method according to claim 30, wherein the tracer comprises fluorescent carbon-based nanoparticles and the said peak luminescence intensity is a peak fluorescence intensity.

34. A method according to claim 30, wherein the carbon-based nanoparticles are doped.

35. A method according to claim 30, wherein the carbon-based nanoparticles are water-dispersible.

36. A method according to claim 30, wherein at least a part of the surface of the carbon-based nanoparticles is hydrophilic.

37. A method according to claim 30, wherein at least a part of the surface of the carbon-based nanoparticles comprises hydrophilic groups, for example selected from one or more of: amine groups, hydroxyl groups, carbonyl groups.
